(19) United States
(12) Patent Application Publication
(10) Pub. No.: US 2009/0304608 A1
(43) Pub. Date: Dec. 10, 2009

(54) Aqueous Hydrogen Peroxide Solution for Use as a Disinfectant or Anti-Microbial Personal Care Product

(75) Inventors: Glenn F. Cueman, Denver, NC (US); Barnwell S. Ramsey, Huntersville, NC (US); Kenneth R. Vest, Davidson, NC (US)

Correspondence Address: Clements Bernard PLLC, 1901 Roxborough Road, Suite 250, Charlotte, NC, 28211, US

(73) Assignee: INNOVASOURCE, LLC, Huntersville, NC (US)

(21) Appl. No.: 12/133,523

(22) Filed: Jun. 5, 2008

(51) Int. Cl. A61K 8/22; A61K 33/40; A61Q 11/00

(52) U.S. Cl. 424/53; 424/616

(57) ABSTRACT

An aqueous hydrogen peroxide solution for use in disinfectants, soaps, shower gels, and mouth rinses is provided, with improved disinfectant and anti-microbial activity. The solutions contain at least one phosphorus based acid compound, e.g. phosphoric and/or a phosphonate and a cationic or non-ionic surfactant. Importantly, the solutions do not require the presence of an anionic surfactant to achieve the objects of the invention, thus enabling successful manufacturing using multiple formulation methods. The solution may also contain fragrances, degreasers, flavors, or other additives depending on the purposes and intended functions of the solutions.

### BACKGROUND OF THE INVENTION

The present invention relates to disinfectant and anti-microbial solutions, and in particular it relates to hydrogen peroxide based solutions with improved disinfectant and antimicrobial properties. More particularly, the present invention relates to hydrogen peroxide solutions in combination with additives of cationic or non-ionic surfactants. Such solutions are used for disinfectant and anti-microbial properties in products used on surfaces and as personal care products such as soaps, shampoos, and mouth rinses.

Most industries and homeowners employ various disinfectants for cleansing of surfaces. There are a plethora of available compounds widely known which can be used to disinfect surfaces. Many of these known compounds and solutions contain peroxygen compounds, chlorine compounds, phenolics, and quaternary ammonium compounds. These compounds and surface agents are known for their germicidal properties and ability to disinfect surfaces with which they come into contact. As such, numerous choices for disinfectants exist.

Despite the large number of widely available compounds and solutions available to consumers for disinfecting surfaces, many are simply impractical for continued use. Indeed, the rate of disinfection is prohibitively slow in many cases, and some compounds and solutions, when used under various conditions, can cause emission of volatile organic compounds. Further complications and detriments to continued use of certain compounds are the tendency for some of these compounds to leave a residue on the surface where it was used or in the environment. Thus a need exists for compounds and solutions that work quickly and do not leave a persistent presence when used.

In addition to disinfecting surfaces, many products now exist that are intended to be used by persons to disinfect a person's skin. Many of these products use anti-microbial compounds to kill germs and bacteria that may be present on skin. Various products use similar type compounds and solutions as the above referenced disinfectants in applications for use on skin. However, use of compounds such as these on skin presents additional concerns and problems that must be overcome. For example, the compounds and solutions must be strong enough to function as a disinfectant/anti-microbial agent, yet be mild enough to use on skin without damaging or irritating the skin. Also, the compounds and solutions must be water soluable and not leave behind any residue when used. Furthermore, the compounds and solutions must be gently enough for continued use on skin, without irritating the skin. Thus there exists a need for compounds and solutions that are effective disinfectants/anti-microbials but gentle enough to be used on skin.

Hydrogen peroxide compounds and solutions have gained favor recently and are currently used in many applications. This is in part due to the low environmental impact of the breakdown products of hydrogen peroxide—water and oxygen. Hydrogen Peroxide is simply water with an extra oxygen molecule (H₂O₂) and breaks down into oxygen and water. Hydrogen Peroxide is produced by both animal and plant cells and is formed naturally in the environment by sunlight acting on water.

Despite its power in high concentrations, hydrogen peroxide is a natural metabolite of many organisms, which decompose the H₂O₂ they produce into oxygen and water. Hydrogen peroxide is also formed by the action of sunlight on water. This results in a natural purification system for the environment. Thus, hydrogen peroxide has none of the problems of gaseous release or chemical residues that are associated with other chemical oxidants. Furthermore, since hydrogen peroxide is totally miscible with water, the safety hazards posed by hydrogen peroxide are only a question of concentration. Industrial strength hydrogen peroxide, a typically high concentration, is a strong oxidizer and as such requires special handling precautions.

Many industrial bleaching operations, such as those used in the production of paper, are increasingly moving towards the use of hydrogen peroxide for a greener bleaching process. It's also the active ingredient in many “oxygen” bleaches and is used extensively for lightening hair. The fact that hydrogen peroxide can inhibit microbial growth (as in the biofouling of water circuits) and encourage microbial growth (as in the bioremediation of contaminated ground waters and soils) shows the wide diversity of applications for which hydrogen peroxide can be used. Similarly, it can treat both easy-to-oxidize pollutants (iron and sulfides) and difficult to oxidize pollutants (solvents, gasolines and pesticides). Hydrogen peroxide can be used for such diverse applications because of the different ways in which its power can be directed, through selectivity. By simply adjusting the conditions of the reaction (e.g., pH, temperature, dose, reaction time, and/or catalyst addition), hydrogen peroxide can be made to oxidize one pollutant over another, or even to favor different oxidation products from the same pollutant.

Also, hydrogen peroxide has been found to have a wide range of antimicrobial properties. In addition, hydrogen peroxide has a broad spectrum of activity, affecting and disinfecting a large range of organisms. Thus, in any disinfectant or anti-microbial, it is important and desirable to have a broad spectrum of activity to effectively deal with situations where harmful organisms are present but their identity is not known.

Despite the apparent advantages of using hydrogen peroxide in a wide range of applications, significant problems with using hydrogen peroxide as a disinfectant have been difficult to overcome. Hydrogen peroxide can be extremely dangerous in a concentrated form. Thus, typical applications utilize a diluted solution of hydrogen peroxide. Indeed, hydrogen peroxide is typically available only up to about a 75% solution of hydrogen peroxide and water, and even this concentration is greatly diluted before many typical uses.

In addition to the physical hazards posed by hydrogen peroxide, it tends to be an unstable compound and will decompose over time. As a result of hydrogen peroxide tending to be unstable and to decompose over time, steps must be taken to preserve and stabilize the hydrogen peroxide solutions, unless the solution is used soon after its creation. Obviously in a commercial setting it is desirable for any disinfectant anti-microbial to have a long shelf life. This is to ensure that if it is to be stored for any length of time, such as on a store shelf before purchase and use by a consumer, it will not degrade and lose its effectiveness. Fortunately, there have developed several ways to improve the stability of hydrogen peroxide compositions thus extending the shelf life of the product. For example, sodium stannate, sodium nitrate, and diethylene triamine penta(methylenephosphonic acid) have been useful as stabilizers, as disclosed in U.S. Pat. No. 5,523,012 to Winterton et al., which issued Jun. 4, 1996. Thus, with the addition of various stabilizing agents, the problems with hydrogen peroxide decomposing have largely been overcome.

In addition to the relatively short shelf life of hydrogen peroxide containing compounds and solutions, another drawback of most disinfectants and anti-microbials is the length of time needed to effectively kill germs and bacteria. Indeed, the amount of time for the compounds and solutions to reduce the bacterial count after the disinfectant has been applied to a bacterially contaminated material has been prohibitively large. For example, it may take 30 minutes or more after application of various disinfectants to disinfect a treated surface. For many applications this time period is simply too large. Certainly in consumer applications it is desirable to use compounds and solutions that effectively disinfect surfaces in much shorter periods of time. Thus, compounds and solutions that kill germs and bacteria in a small amount of time are needed.

As research and development into utilizing hydrogen peroxide as a disinfectant and anti-microbial has progressed, some of the developed compounds utilizing hydrogen peroxide as a disinfectant or anti-microbial have overcome many of these associated problems. Indeed, it has been discovered that the addition of a surfactant to hydrogen peroxide based compounds and solutions is desirable. For example, Winterton et al. discloses, in U.S. Pat. No. 5,523,012, a buffered, hydrogen peroxide based, disinfecting solution for contact lenses. Winterton discloses a formulation that contains about 0.1% to about 1.0% by weight of an ocularly compatible surfactant. Winterton disclosed that in one experiment, addition of about 0.4% anionic sulpho-succinate surfactant improved the killing time for aspergillus furnigatus to 6.9 minutes, compared to 9.4 minutes for a solution containing 0.1% nonionic surfactants. Thus, it seems that the addition of surfactants can further reduce the time needed to properly disinfect the surface and kill harmful microbes. As such further research and development regarding increasing the effectiveness and speed of hydrogen peroxide based compounds and anti-microbials is needed.

In the current state of the art, it has been found that addition of phosphorus-based acids and anionic surfactants greatly enhance the activity of aqueous hydrogen peroxide solutions thereby reducing the time needed to effectively disinfect a surface. In other words, addition of phosphorus-based acids can increase the rate at which the solutions kill germs and bacteria. These phosphorus-based acids used in the compounds and solutions can be inorganic acids or organic acids. Especially preferred are phosphoric acid (H₃PO₄) and phosphonates having 1 to 5 phosphonic acid groups. Particularly preferred phosphonates are amino tri(methylene phosphonic acid), 1-hydroxyethylidene-1,1-diphosphonic acid, diethylenetriaminepenta-(methylene phosphonic acid), 2-hydroxyethylimino bis(methylene phosphonic acid), ethylene diamine tetra(methylene phosphonic acid). Any of these acids may be used alone as an additive to hydrogen peroxide solutions, but mixtures of phosphoric acid and at least one of the phosphonates seem to provide more advantageous results. Some of these phosphonic acids are available from Albright & Wilson under the trademark BRIQUEST and some from Solutia, Inc., under the trademark DEQUEST. The concentration of the phosphorus-based acids is lowered for solutions with lower concentrations of hydrogen peroxide. The pH of the solutions can be adjusted as well.

For example, U.S. Pat. No. 6,346,279 to Rochon discloses a formulation for hydrogen peroxide based solutions that significantly reduces the time required for disinfecting a surface. Rochon teaches the use of phosphorus-based acids and an anionic surfactant in combination with hydrogen peroxide to create an effective disinfectant and anti-microbial solution. Rochon discloses a solution of only about 0.5% (w/w) hydrogen peroxide that has a shelf life of up to a year or more and an effective disinfecting reaction time of as little as 30 seconds. Thus, it seems that many of the problems associated with hydrogen peroxide based disinfecting solutions have been overcome. However, the necessary use of anionic surfactants as an additive to hydrogen peroxide based solutions greatly limits the formulation options available to create and manufacture the disinfectants and anti-microbials. It is thus well known in the art and advantageous to use surfactants in hydrogen peroxide containing disinfectants and anti-microbial solutions.

Use of surfactants in disinfectants and anti-microbials is well known. Surfactants typically contain both hydrophobic groups in their “tails” and hydrophilic groups in their “heads.” This allows the surfactant to dissolve in both organic solvents and non-organic solvents. Thus, a surfactant reduces the interfacial tension between oil and water by adsorbing at the liquid-liquid interface. Thus, in surface applications, a surfactant can allow the disinfectant anti-microbial to interact with both inorganic and organic compounds. This is due to a surfactant forming aggregates. Examples of such aggregates are vesicles and micelles. The concentration at which surfactants begin to form micelles is known as the critical micelle concentration. When micelles form in water, their tails form a core that can encapsulate an oil droplet, and their (ionic/polar) heads form an outer shell that maintains favorable contact with water (an inorganic solvent). When surfactants assemble in oil (or other organic solvent), the aggregate is referred to as a reverse micelle. In a reverse micelle, the heads are in the core and the tails maintain favorable contact with oil. The ability of a surfactant to interact with both oil (organic) and water (non-organic) solutions is fundamental to creating an effective disinfectant and anti-microbial since surfaces may contain both organic and non-organic material. Thus, it is desirable to use a surfactant in disinfectants and anti-microbial compounds and solutions.

Surfactants are typically classified into four primary groups; anionic, cationic, non-ionic, and zwitterionic (dual charge). Which classification a surfactant belongs in is determined by the presence of formally charged groups in its head. A non-ionic surfactant has no charge groups in its head. The head of an ionic surfactant carries a net charge. If the charge is negative, the surfactant is anionic; if the charge is positive, it is cationic. If, however, a surfactant contains a head with two oppositely charged groups, it is termed zwitterionic.

In the realm of disinfectant and anti-microbial compounds and solutions, some commonly encountered anionic (negatively charged) surfactants are based on sulfate, sulfonate or carboxylate anions and include: sodium dodecyl sulfate (SDS), ammonium lauryl sulfate and other alkyl sulfate salts, sodium laureth sulfate (also known as sodium lauryl ether sulfate (SLES)), alkyl benzene sulfonate, and soaps or fatty acid salts.

In contrast, some commonly encountered cationic (positively charged) surfactants can be based on quaternary ammonium cations and include: cetyl trimethylammonium bromide (CTAB) a.k.a. hexadecyl trimethyl ammonium bromide and other alkyltrimethylammonium salts, cetylpyridinium chloride (CPC), polyethoxylated tallow amine (POEA), benzalkonium chloride (BAC), and benzethonium chloride (BZT).

Further, some commonly encountered zwitterionic (amphoteric) surfactants include: dodecyl betaine, dodecyl dimethylamine oxide, cocamidopropyl betaine, and coco ampho glycinate.

Unlike anionic and ionic surfactants, cationic surfactants are particularly suited for use in applications that are applied to skin. Cationic surfactants are less harsh than anionic and ionic surfactants, and therefore they tend to reduce the chance for skin to become irritated. Indeed, cationic surfactants are a skin friendly additive. As such, there is a desire in the antimicrobial/disinfectant field for additives that can kill bacterial but are gentle enough to use on skin.

Hydrogen peroxide and cationic surfactants are generally compatible in solution. In addition to hydrogen peroxide possessing antimicrobial/disinfecting, properties, cationic surfactants also display the ability to act as antimicrobials and disinfectants. The cationic surfactants are able to kill bacteria and germs much like hydrogen peroxide. Cationic surfactants also act as stabilizers for the hydrogen peroxide, slowing down the process by which hydrogen peroxide degrades. However, tie degradation of hydrogen peroxide is the process by which hydrogen peroxide is able to disinfect and kill bacteria. As the hydrogen peroxide breaks down, it generates free radicals that kill bacteria. The rate at which the free radicals kill the bacteria is directly proportional to the rate at which the hydrogen peroxide breaks down. Thus, the faster the hydrogen peroxide degrades, the faster the bacteria are killed. This present a problem however, because once the hydrogen peroxide is totally degraded, the solution loses its antimicrobial properties. As such, it is desirable to stabilize the hydrogen peroxide and slow down the rate at which it degrades. However, this lessens the strength of the bacteria killing properties of the solution.

The answer to this problem of degradation lies in the use of cationic surfactants. Since cationic surfactants possess antimicrobial characteristics of their own, the more cationic surfactant present in the solution, the more bacteria are killed. Additionally, the cationic surfactant tends to stabilize the hydrogen peroxide and reduce the rate at which it breaks down. Use of cationic surfactants in the solution has a dual benefit—it preserves the hydrogen peroxide and it kills bacteria. Thus, the concentration of hydrogen peroxide in an antimicrobial/disinfectant solution can be lower if more cationic surfactant is present. Therefore, concentrations of surfactant and hydrogen peroxide can be varied while still meeting a minimum acceptable rate of killing bacteria.

Thus, the present invention is directed to improving the efficiency of hydrogen peroxide based compounds and solutions for use as a disinfectant and/or anti-microbial solution. The present invention is further directed to hydrogen peroxide based solutions containing cationic surfactants, and improving the formulation options for their manufacture.

### BRIEF SUMMARY OF THE INVENTION

Accordingly, the present invention provides an aqueous solution for use as a disinfectant and anti-microbial agent. More specifically, the present invention provides a solution of water with hydrogen peroxide and a phosphorus-based acid. Further the present invention also provides a solution containing a surfactant. Importantly, the present invention does not require the presence of an anionic surfactant. The concentrations of these and other components can be varied dependant upon the end use contemplated for the solution.

The present invention provides a hydrogen peroxide based, aqueous solution for use as a disinfectant and/or an anti-microbial. The solution contains hydrogen peroxide (35%), generally in a concentration range of between 2.0 to 9.0% w/w, a phosphorus-based acid generally in a concentration range of between 0.03 and 0.70% w/w, and at least one cationic or non-ionic surfactant generally in a concentration range of between 0.04 and 0.07% w/w.

The present invention further provides a hydrogen peroxide based, aqueous solution for personal care products such as hand washes, shampoos, shower gels, soaps, shower foams and mouth rinses. The personal care products using hydrogen peroxide based solutions must be and are suitable for use on skin. Accordingly, the present invention as used in personal care products contains hydrogen peroxide generally in a concentration range of between 1.0 and 3.5% w/w, a phosphorus-based acid generally in a concentration range of between 0.30 and 0.40% w/w, and at least one cationic or non-ionic surfactant generally in a concentration range of between 0.04 and 0.07% w/w.

The present invention still further provides a hydrogen peroxide based, aqueous solution for use as a disinfectant on various surfaces. The disinfectant solution contains hydrogen peroxide (50%), generally in a concentration range of between 3.0 and 6.1% w/w, a phosphorus-based acid (phosphoric acid 85%) generally in a concentration range of between 0.30 and 0.40% w/w, and at least one cationic or non-ionic surfactant generally in a concentration range of between 0.04 and 0.07% w/w. In the disinfectant solution, the concentration of hydrogen peroxide can be higher than in the personal care products because there is less likely to be contact with skin leading to irritation or damage. More specifically, the disinfectant has a concentration of hydrogen peroxide generally in a concentration range of between 5.9 and 6.1% w/w.

The present invention also provides a solution for use in personal care products. In addition to the hydrogen peroxide, phosphoric acid, and cationic or non-ionic surfactant, the solution may contain various concentrations of deionized water generally between 30.00 and 84.00% w/w, cocamidopropyl betaine between 0.05 and 35.00% w/w. The concentration of hydrogen peroxide (35%) is between 2.00 and 9.00% w/w and there is also methyl gluceth-20 between 0.50 and 6.00% w/w. Further, the invention provides aloe gel between 0.05 and 1.00% w/w, polyaminopropyl biguanide between 0.10 and 1.50% w/w, a fragrance between 0.05 and 1.00% w/w, phosphoric acid (85%) between 0.10 and 0.70% w/w, disodium EDTA between 0.02 and 0.30% w/w, and lauramine oxide between 2.00 and 9.00% w/w.

The present invention also provides a solution for use in personal care products. In addition to the hydrogen peroxide, phosphoric acid, and cationic or non-ionic surfactant, the solution may contain various concentrations of deionized water generally between 30.00 and 84.00% w/w, cocamidopropyl betaine between 0.05 and 35.00% w/w. The concentration of hydrogen peroxide (35%) is between 2.00 and 9.00% w/w and there is also methyl gluceth-20 between 0.50 and 6.00% w/w. Further, the invention provides aloe gel between 0.05 and 1.00% w/w, polyaminopropyl biguanide between 0.10 and 1.50% w/w, a fragrance between 0.05 and 1.00% w/w, phosphoric acid (85%) between 0.10 and 0.70% w/w, disodium EDTA between 0.02 and 0.30% w/w, and lauramine oxide between 2.00 and 9.00% w/w. The present invention further contains a solution containing panthenol between 0.01 and 2.5% w/w.

The present invention further provides a solution comprising lauramide diethanolamide between 0.50 and 6.00% w/w and PEG-120 methyl glucose dioleate between 0.20 and 1.00% w/w.

The present invention further provides a solution for use in personal care products. In addition to the hydrogen peroxide, phosphoric acid, and cationic or non-ionic surfactant, the solution may contain various concentrations of deionized water generally between 30.00 and 84.00% w/w, cocamidopropyl betaine between 0.05 and 35.00% w/w. The concentration of hydrogen peroxide (35%) is between 2.00 and 9.00% w/w and there is also methyl gluceth-20 between 0.50 and 6.00% w/w. Further, the invention provides aloe gel between 0.05 and 1.00% w/w, polyaminopropyl biguanide between 0.10 and 1.50% w/w, a fragrance between 0.05 and 1.00% w/w, phosphoric acid (85%) between 0.10 and 0.70% w/w, disodium EDTA between 0.02 and 0.30% w/w, and lauramine oxide between 2.00 and 9.00% w/w. Also included in the solution is Phospholipid PTC between 0.5 and 2.5% w/w.

The present invention still further provides a solution further comprising lauramide diethanolamide between 0.50 and 6.00% w/w and PEG-120 methyl glucose dioleate between 0.20 and 1.00% w/w.

Further, the present invention contemplates and provides a solution wherein the hydrogen peroxide is in the form of hydrogen peroxide polyvinyl pyrrolidone complex containing 20% w/w hydrogen peroxide and having a weight average molecular weight of 58,000, in a concentration of from 10.0 to 20.0% w/w wherein the solvent is polyoxyethylene sorbitan monolaurate in a concentration of from 0.20 to 0.35% w/w.

The present invention further provides a solution wherein the hydrogen peroxide is in the form of hydrogen peroxide polyvinyl pyrrolidone complex containing 20% w/w hydrogen peroxide and having a weight average molecular weight of 58,000, in a concentration of from 10.0 to 20.0% w/w wherein tie solvent is polyoxyethylene sorbitan monolaurate in a concentration of from 0.20 to 0.35% w/w, and the surfactant is cocamidopropyl betaine in a concentration of from 0.01 to 0.10% w/w.

The present invention still further provides a solution farther containing flavoring in a concentration of from 0.10 to 2.50% w/w %, and a sweetener in a concentration of from 0.01 to 0.10% w/w.

The present invention also provides a mouth rinse wherein the flavoring is a blend of peppermint flavor and orange flavor.

Further, the present invention provides a solution for use as a mouth rinse wherein the solution is: 80.00% to 85% deionized water, 13.00 to 17.00% peroxydone K30, 0.01% to 0.10% cocamidopropyl betaine, 0.20% to 0.60% sweetener, 0.75% to 2.00% peppermint flavor, 0.10% to 0.28% orange flavor, 0.10% to 0.40% polyoxyethylene sorbitan monolaurate, and 0.03% to 0.05% phosphoric acid.

The present invention also provides a disinfectant further comprising: lauramine oxide in a concentration of 0.40 to 0.50% w/w, a solvent in a concentration of 85 to 90% w/w, a degreaser in a concentration of 0.01 to 0.03% w/w, a fragrance in a concentration of 0.085 to 0.15% w/w, and an anionic surfactant in a concentration of 0.01 to 0.03% w/w.

Still further the present invention provides a disinfectant wherein the anionic surfactant is sodium 1-octane sulfonate in a concentration of 0.01 to 0.03% W/W, and the solvent is polyoxyethylene sorbitan monolaurate in a concentration of 0.03 to 0.04% w/w.

The present invention further provides a disinfectant wherein the degreaser is Steposol DG in a concentration of 0.01 to 0.03% w/w and the phosphoric acid (85%) is in a concentration of 0.03 to 0.04% w/w.

Still further, the present invention provides a method of making a quantity of hydrogen peroxide based mouth rinse comprising: mixing 80 to 85% w/w deionized water with 10 to 20% w/w of Peroxydone K₃₀; stirring said mixture until the solution is substantially clear; adding 0.01 to 0.10% w/w cocamidopropyl betaine, 0.01 to 0.10% w/w sorbatame, and 0.03 to 0.05% w/w phosphoric acid (85%) to tie solution; stirring the solution; separately mixing 0.10 to 2.50% w/w flavoring and 0.20 to 0.35% w/w polyoxyelthelene sorbitan monolaurate under continual stirring; adding the flavoring solution to the main solution under continual stirring; diluting the solution with deionized water until 100% w/w of the solution is reached.

Thus, it can be readily seen that the present invention provides useful hydrogen peroxide based solutions that contain a phosphorus based acid, a cationic or non-ionic surfactant, and possess disinfectant and anti-microbial properties. The present invention discloses and teaches hydrogen peroxide solutions that can be used in a wide variety of applications, and in particular hand washes, bath/shower gels, bath/shower foams, disinfectants, and mouth rinses.

### DETAILED DESCRIPTION OF THE PREFERRED EMBODIMENT

The present invention provides a solution that is useful as a hand sanitizing wash gel in a preferred embodiment consisting of various components. This preferred embodiment of the present invention includes a solution of deionized water generally in a concentration range of between 30.00 and 80.00% w/w, cocamidopropyl betain (an amphoteric surfactant) generally in a concentration range of between 5.00 and 35.00% w/w, a solution of 35% hydrogen peroxide generally in a concentration range of between 2.00 and 9.00% w/w, methyl gluceth-20 generally in a concentration range of between 0.50 and 6.00% w/w, aloe gel generally in a concentration range of between 0.05 and 1.00% w/w, polyaminopropyl biguanide (preservative) generally in a concentration range of between 0.10 and 1.50% w/w, a fragrance (Springfresh) generally in a concentration range of between 0.05 and 1.00% w/w, phosphoric acid 85% (pH 4.0) generally in a concentration range of between 0.10 and 0.70% w/w, disodium EDTA (a sequestrant) generally in a concentration range of between 0.04 and 0.30% w/w, PEG-120 methyl glucose dioleate generally in a concentration range of between 0.20 and 1.00% w/w, lauric diethanolamide generally in a concentration range of between 0.50 and 6.00% w/w, lauramine oxide generally in a concentration range of between 2.00 and 9.00% w/w, and Phospholipid PTC (diester phosphatides) generally in a concentration range of between 0.50 and 2.50% w/w. Such preferred embodiment is useful as a hand sanitizing wash gel, effectively killing germs and bacteria in a timely manner without irritating or damaging skin.

The present invention provides a solution that is useful as a hand sanitizing wash foam in a preferred embodiment consisting of various components. This preferred embodiment of the present invention includes a solution of deionized water generally in a concentration range of between 40.00 and 83.00% w/w, cocamidopropyl betain (an amphoteric surfactant) generally in a concentration range of between 5.00 and 35.00% w/w, a solution of 35% hydrogen peroxide generally in a concentration range of between 2.00 and 9.00% w/w, methyl gluceth-20 generally in a concentration range of between 0.50 and 6.00% w/w, aloe gel generally in a concentration range of between 0.05 and 1.00% w/w, polyaminopropyl biguanide (preservative) generally in a concentration range of between 0.30 and 1.20% w/w, a fragrance (Springfresh) generally in a concentration range of between 0.05 and 1.00% w/w, phosphoric acid 85% (pH 4.0) generally in a concentration range of between 0.10 and 0.70% w/w, disodium EDTA (a sequestrant) generally in a concentration range of between 0.02 and 0.40% w/w, lauramine oxide generally in a concentration range of between 2.00 and 9.00% w/w, and Phospholipid PTC (diester phosphatides) generally in a concentration range of between 0.50 and 2.50% w/w. Such preferred embodiment is useful as a hand sanitizing wash foam, effectively killing germs and bacteria in a timely manner without irritating or damaging skin.

Another preferred embodiment of the present invention provides a bath or shower foam useful as an anti-microbial wash. This preferred embodiment of the present invention includes a solution of deionized water generally in a concentration range of between 40.00 and 83.00% w/w, cocamidopropyl betain (an amphoteric surfactant) generally in a concentration range of between 5.00 and 35.00% w/w, a solution of 35% hydrogen peroxide generally in a concentration range of between 2.00 and 9.00% w/w, methyl gluceth-20 generally in a concentration range of between 0.50 and 6.00% w/w, aloe gel generally in a concentration range of between 0.05 and 1.00% w/w, polyaminopropyl biguanide (preservative) generally in a concentration range of between 0.10 and 1.50% w/w, a fragrance (Springfresh) generally in a concentration range of between 0.05 and 1.00% w/w, phosphoric acid 85% (pH 4.0) generally in a concentration range of between 0.10 and 0.70% w/w, disodium EDTA (a sequestrant) generally in a concentration range of between 0.04 and 0.30% w/w, lauramine oxide generally in a concentration range of between 2.00 and 9.00% w/w, and panthenol generally in a concentration range of between 0.01 and 2.50% w/w. Such preferred embodiment is useful as a bath shower foam, effectively killing germs and bacteria in a timely manner without irritating or damaging skin.

The present invention further provides a bath and shower gel in a preferred embodiment useful as an anti-microbial wash. The present invention provides a solution that is useful as a shower gel in a preferred embodiment consisting of various components. This preferred embodiment of the present invention includes a solution of deionized water generally in a concentration range of between 30.00 and 84.00% w/w, cocamidopropyl betain (an amphoteric surfactant) generally in a concentration range of between 5.00 and 35.00% w/w, a solution of 35% hydrogen peroxide generally in a concentration range of between 2.00 and 9.00% w/w, methyl gluceth-20 generally in a concentration range of between 0.50 and 6.00% w/w, aloe gel generally in a concentration range of between 0.05 and 1.00% w/w, polyaminopropyl biguanide (preservative) generally in a concentration range of between 0.10 and 1.50% w/w, a fragrance (Springfresh) generally in a concentration range of between 0.05 and 1.00% w/w, phosphoric acid 85% (pH 4.0) generally in a concentration range of between 0.10 and 0.70% w/w, disodium EDTA (a sequestrant) generally in a concentration range of between 0.04 and 0.30% w/w, PEG-120 methyl glucose dioleate generally in a concentration range of between 0.20 and 1.00% w/w, lauric diethanolamide generally in a concentration range of between 0.50 and 6.00% w/w, lauramine oxide generally in a concentration range of between 2.00 and 9.00% w/w, and panthanol generally in a concentration range of between 0.01 and 2.50% w/w. Such preferred embodiment is useful as a shower gel, effectively killing germs and bacteria in a timely manner without irritating or damaging skin.

The present invention still further provides a hydrogen peroxide based mouth rinse in a preferred embodiment useful as an oral disinfectant. This preferred embodiment of the present invention includes a solution of: 78.00 to 84.00% w/w deionized water, 11.00 to 9.00% w/w hydrogen peroxide polyvinyl pyrrolidone complex containing 20% w/w hydrogen peroxide and having a weight average molecular weight of 58,000 (available as Peroxydone K-30, produced by ISP Corporation), 0.01 to 0.10% w/w cocamidopropyl betaine, 0.10 to 0.90% w/w sweetener, 0.0 to 2.00% w/w peppermint flavor, 0.0 to 0.50% w/w orange flavor, 0.15 to 0.50% w/w polyoxyethylene sorbitan monolaurate, and 0.01 to 0.10% w/w phosphoric acid.

The present invention still further provides a hydrogen peroxide based disinfectant useful on surfaces according to the following preferred embodiment. A solution of between 85.0 and 90.0% w/w purified water, 3.0 to 6.1% w/w hydrogen peroxide (50%), 0.040 to 0.050% w/w of Lauramine Oxide (30% active), 0.01 to 0.03% w/w degreaser comprising ammonium methyl sulfate (40%), fatty alcohol ethoxylate (40%), and stepsol DG, a 0.01 to 0.03% w/w surfactant comprising Bio-Terge® (sodium octanesulfonate (35%), sodium sulfate (3%), isopropanol (3%)), between 0.085 and 0.150% w/w of a fragrance, between 0.030 and 0.040% w/w of a solvent (polyoxyethylene sorbitan monolaurate “Tween 20”), and 0.030 and 0.040% w/w phosphoric acid.

The present invention has been tested yielding unexpected, surprising results. The efficiency of the present invention's hydrogen peroxide based solution utilizing a phosphorus based acid and cationic or non-ionic surfactants in lieu of anionic surfactants at reducing bacteria is quite high. Indeed, according to the testing, the solutions prepared with cationic surfactants were at least as effective at killing bacteria as were the solutions utilizing anionic surfactants. By means of a representative example, a solution of the mouth rinse was prepared, and its efficiency in killing bacteria was tested.

The mouth rinse solution according to the present invention was prepared for testing purposes according to the following method. A 200 gram sample size was prepared by first placing 150 g of deionized water into a reaction vessel. Next, 30 g of Peroxydone K30 was slowly added while continually mixing the reaction vessel containing the mixture. Mixing of these components was continued until the solution had obtained a clear appearance. At this point, the cocamidopropyl betaine, sorbatame, and phosphoric acid were added to the reaction vessel and mixed. In a separate mixing container, the peppermint flavoring, orange flavoring, and Tween 20 were added under constant mixing. Once this mixture was adequately blended, it was introduced into the main reaction vessel. Finally, additional deionized water was added to the reaction vessel until the solution volume was equal to 200 ml. This preferred embodiment of the present invention was used in testing.

In addition to the mouth rinse, a hydrogen peroxide based disinfectant solution was prepared according to the present invention for purposes of testing the disinfectant and anti-microbial properties of the invention. To prepare this preferred embodiment of the present invention, purified water was added to a reaction vessel and a solution of 50% hydrogen peroxide was introduced into the reaction vessel and mixed with the water. Lauramine oxide (30% active, available as Ammonyx® LO from Stepan Co.) was added to the reaction mixture. Next, ammonium methyl sulfate and fatty alcohol ethoxylate (40% and 40%, available as Stepsol DG from Stepan Co.) was added to the reaction vessel and mixed with the solution. Next, a solution of sodium 1-octane sulfonate (35%) (available as Bio-Terge® PAS-SS from Stepan Co.), sodium sulfate (3%), and isopropanol (3%), was added to the reaction vessel and mixed with the disinfectant solution. A fragrance (Item 31217 from Chemia Corp.) followed by a solvent (Tween 20 available from Unigema Croda, Inc.) was added to the solution. Next, phosphoric acid (85%) was added to the reaction vessel and mixed with the disinfectant solution. Finally, additional purified water was added and mixed with the disinfectant solution such that the concentrations of the components comprising the disinfectant solution were as follows: purified water=87.575% w/w, hydrogen peroxide (50%)=6% w/w, lauramine oxide=0.15% w/w, Steposol® DG=0.05% w/w, Bio-Terge® PAS-8S=0.05% w/w, fragrance=0.1% w/w, Tween 20=0.035% w/w, and phosphoric acid (85%)=0.04% w/w.

These samples were tested to determine and compare the efficiency of the solutions' disinfectant and anti-microbial properties of the present invention. The solutions were prepared according to the above described methods and sample sizes of 10 ml were used. Bacterial test cultures used were Escherichia coli and Staphylococcus aureus. The bacteria cultures were prepared in solutions containing 10⁸ CFU/ml of bacteria cultures. The mouti rinse solution and the disinfectant solution were place in separate, sterile flasks in 10 ml quantities. The mouth rinse and disinfectant solutions were inoculated with 1 ml of the bacteria cultures and marked at “Time 0 minutes.” The inoculated mouth rinse and disinfectant solutions were then placed in a platform shaker for 10 minutes and then removed. The mouth rinse and disinfectant solutions were then tested at “Time 10 minutes.” Results for the tests are shown in Table 1.

TABLE 1

| | | | | |
|---|---|---|---|---|
| | Time | Time | | |
| | 0 Minutes | 10 Minutes | | |
| | Test | Number of | | |
| | Population | Survivors | | Log |
| Test Culture | (CFU/ml) | (CFU/ml) | % Reduction | Reduction |

Mouth Rinse

| | | | | |
|---|---|---|---|---|
| Escherichia coli | 1.05 × 10⁷ | <10 | >99.9999% | 6.02 Log¹⁰ |
| Staphylococcus | 1.20 × 10⁷ | <10 | >99.9999% | 6.08 Log¹⁰ |
| aureus | | | | |

Disinfectant

| | | | | |
|---|---|---|---|---|
| Escherichia coli | 1.05 × 10⁷ | <10 | >99.9999% | 6.02 Log¹⁰ |
| Staphylococcus | 1.20 × 10⁷ | <10 | >99.9999% | 6.08 Log¹⁰ |
| aureus | | | | |

As shown in Table 1, testing indicates that the present invention possesses superior disinfecting and anti-microbial properties, killing in excess of 99.9999% of both bacteria cultures. Both solutions achieved a minimum of a 6 log reduction for both bacteria cultures tested after only 10 minutes. Thus, the invention achieves its goals of providing an effective disinfectant/anti-microbial that is capable of working in a short amount of time.

Thus, it can readily be seen from the foregoing empirical testing data that the present invention provides an effective disinfectant and anti-microbial, hydrogen peroxide based solution without the presence of a substantial amount of an anionic surfactant. The test results show that the present invention possesses disinfecting and anti-microbial properties similar to or greater than other hydrogen peroxide based solutions. Also shown by the test results is that the present invention's use of a cationic surfactant (embodied in the mouth rinse) does not impede the effectiveness of the solution. Indeed, the concentration of hydrogen peroxide in the mouth rinse is only half of that in the disinfectant (3% versus 6%) and yet the mouth rinse performed identically to the disinfectant. Both killed 99.9999% of the bacteria in only 10 minutes. Thus it is clear that the present invention has overcome the problem of hydrogen peroxide based disinfectants and anti-microbials requiring the use of anionic surfactants. The present invention overcomes this limitation by providing effective disinfectant and anti-microbial solutions without requiring substantial quantities of anionic surfactants and without compromising the effectiveness of the disinfectant/anti-microbial.

It should be understood that the embodiments described herein are for illustration purposes only, and the present invention is not limited to the solutions described herein. Rather, the present invention can be readily adaptable for use in many different forms, all of which are intended to be covered.

What is claimed is:

1. A hydrogen peroxide based, aqueous solution for use as a disinfectant and/or an anti-microbial comprising: i) an aqueous hydrogen peroxide solution in a concentration of from 0.007% to 0.032% w/w, ii) at least one phosphorus-based acid solution in a concentration of from 0.00025 to 0.00595% w/w, and iii) at least one cationic or non-ionic surfactant in a concentration of from 0.05 to 35.0% w/w.

2. A solution according to claim 1 further comprising: a solution of deionized water between 30.00 and 84.00% w/w, cocamidopropyl betaine between 0.05 and 35.00% w/w, hydrogen peroxide (35%) between 2.00 and 9.00% w/w, methyl gluceth-20 between 0.50 and 6.00% w/w, aloe gel between 0.05 and 1.00% w/w, polyaminopropyl biguanide between 0.10 and 1.50% w/w, a fragrance between 0.05 and 1.00% w/w, phosphoric acid (85%) between 0.10 and 0.70% w/w, disodium EDTA between 0.02 and 0.30% w/w, and lauramine oxide between 2.00 and 9.00% w/w.

3. A solution according to claim 2 further comprising panthenol between 0.01 and 2.5% w/w.

4. A solution according to claim 3 further comprising lauramide diethanolamide between 0.50 and 6.00% w/w and PEG-120 methyl glucose dioleate between 0.20 and 1.00% w/w.

5. A solution according to claim 2 further comprising Phospholipid PTC between 0.5 and 2.5% w/w.

6. A solution according to claim 5 further comprising lauramide diethanolamide between 0.50 and 6.00% w/w and PEG-120 methyl glucose dioleate between 0.20 and 1.00% w/w.

7. A solution according to claim 1 wherein the hydrogen peroxide is in the form of hydrogen peroxide polyvinyl pyrrolidone complex containing 20% w/w hydrogen peroxide and having a weight average molecular weight of 58,000, in a concentration of from 10.0 to 20.0% w/w and the solvent is polyoxyethylene sorbitan monolaurate in a concentration of from 0.20 to 0.35% w/w.

8. A solution according to claim 7 wherein the surfactant is cocamidopropyl betaine in a concentration of from 0.01 to 0.10% w/w.

9. A solution according to claim 8 further comprising flavoring in a concentration of from 0.10 to 2.50% w/w %, and a sweetener in a concentration of from 0.01 to 0.10% w/w.

10. A solution for use as a mouth rinse according to claim 9 wherein the flavoring is a blend of peppermint flavor and orange flavor.

11. A solution for use as a mouth rinse according to claim 10 wherein the solution is: 80.00% to 85% deionized water, 13.00 to 17.00% peroxydone K30, 0.01% to 0.10% cocamidopropyl betaine, 0.20% to 0.60% sweetener, 0.75% to 2.00% peppermint flavor, 0.10% to 0.28% orange flavor, 0.10% to 0.40% polyoxyethylene sorbitan monolaurate, and 0.03% to 0.05% phosphoric acid.

12. A disinfectant according to claim 1 further comprising: lauramine oxide in a concentration of 0.40 to 0.50% w/w, a solvent in a concentration of 85 to 90% w/w, a degreaser in a concentration of 0.01 to 0.03% w/w, a fragrance in a concentration of 0.085 to 0.15% w/w, and an anionic surfactant in a concentration of 0.01 to 0.03% w/w.

13. A disinfectant according to claim 12 wherein the anionic surfactant is sodium 1-octane sulfonate in a concentration of 0.01 to 0.03% w/w, and the solvent is polyoxyethylene sorbitan monolaurate in a concentration of 0.03 to 0.04% w/w.

14. A disinfectant according to claim 13 wherein the degreaser is Steposol DG in a concentration of 0.01 to 0.03% w/w and the phosphoric acid (85%) is in a concentration of 0.03 to 0.04% w/w.

15. A method of making a quantity of hydrogen peroxide based mouth rinse comprising: mixing 80 to 85% w/w deionized water with 10 to 20% w/w of Peroxydone K30; stirring said mixture until the solution is substantially clear; adding 0.01 to 0.10% w/w cocamidopropyl betaine, 0.01 to 0.10% w/w sorbatame, and 0.03 to 0.05% w/w phosphoric acid (85%) to the solution; stirring the solution; separately mixing 0.10 to 2.50% w/w flavoring and 0.20 to 0.35% w/w polyoxyelthelene sorbitan monolaurate under continual stirring; adding the flavoring solution to the main solution under continual stirring; diluting the solution with deionized water until 100% w/w of the solution is reached.
